(19) United States
(12) Patent Application Publication
(10) Pub. No.: US 2025/0312570 A1
(43) Pub. Date: Oct. 9, 2025

(54) Staple-Actuated Catheter Fixation Device

(71) Applicant: Abiomed, Inc., Danvers, MA (US)

(72) Inventor: Christopher Sheils, Danvers, MA (US)

(73) Assignee: Abiomed, Inc., Danvers, MA (US)

(21) Appl. No.: 19/087,722

(22) Filed: Mar. 24, 2025

Related U.S. Application Data
(60) Provisional application No. 63/575,311, filed on Apr. 5, 2024.

(51) Int. Cl. A61M 25/04

(52) U.S. Cl. CPC A61M 25/04

(57) ABSTRACT

A catheter fixation device for securing a catheter within a cardiovascular system of a patient. The catheter fixation device includes a body formed of biocompatible material arranged to be subcutaneously secured within a patient, the body including a catheter receiving portion defining a cavity, and first and second wings extending from the catheter receiving portion. The first and second wings are moveable relative to one another from an unclamped position, in which a catheter is moveable within the catheter receiving portion, and a clamped position, in which the catheter is fixed within the catheter receiving portion. A method of delivering and stabilizing a medical device within a cardiovascular system of a patient is also provided.

### CROSS-REFERENCE TO RELATED APPLICATIONS

This application claims the benefit of the filing date of U.S. Provisional Patent Application No. 63/575,311 filed Apr. 5, 2024, the disclosure of which is hereby incorporated herein by reference.

### BACKGROUND OF THE DISCLOSURE

The present disclosure relates to catheter fixation devices, and more particularly, to a fixation device that allows a physician to subcutaneously stabilize a catheter within the cardiovascular system of a patient.

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the IMPELLA® family of devices designed by Abiomed, Inc. of Danvers, MA. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY OF THE DISCLOSURE

In accordance with a first aspect of the present disclosure, a catheter fixation device is provided. Among other advantages, the catheter fixation device is designed to be subcutaneously implanted into a patient and utilized to secure a catheter carrying an intracardiac pump. Securing the catheter subcutaneously reduces the distance between the securement device and the target site (e.g., across a native heart valve) compared to traditional securement devices which are externally secured to skin of the patient. As a result, the subcutaneous catheter fixation device disclosed herein, reduces the length between the catheter fixation device and the distal end of the catheter, which in turn reduces the likelihood of migration of the catheter compared to the traditional catheter fixation devices.

One embodiment of the catheter fixation apparatus includes a body formed of biocompatible material arranged to be subcutaneously secured within a patient. The body includes a catheter receiving portion defining a cavity, and first and second wings extending from the catheter receiving portion. The first and second wings are moveable relative to one another from an unclamped position, in which a catheter is moveable within the catheter receiving portion, and a clamped position, in which the catheter is fixed within the catheter receiving portion.

The body may include an inner layer formed of a substantially resilient material and an outer layer formed of a substantially rigid material. In one aspect, the substantially resilient material may be a polymeric material and/or the outer layer may be a metal or metal alloy. Advantageously, the outer layer may define one or more apertures arranged to receive a closure device, such as a staple, to secure the first and seconds wings in the clamped position.

A sheath assembly is also provided herein and includes a catheter for delivering a medical device, such as an intracardiac pump, into a cardiovascular system of a patient, and a catheter fixation device including a body formed of a biocompatible material arranged to be subcutaneous secured within the patient. The body includes a catheter receiving portion defining a cavity, and first and second wings extending from the catheter receiving portion. The first and second wings are moveable relative to one another from an unclamped position, in which the catheter is moveable within the catheter receiving portion, and a clamped position, in which the catheter is fixed within the catheter receiving portion.

The body may include an inner layer formed of a substantially resilient material and an outer layer formed of a substantially rigid material. In one aspect, the substantially resilient material may be a polymeric material, and the substantially rigid material may be a metal or metal alloy. Again, the outer layer may define an aperture arranged to receive a closure device such as a staple or a suture.

A method of stabilizing a medical device within a cardiovascular system of a patient is also provided herein and includes the step of: tracking a catheter, carrying a medical device, through the cardiovascular system of the patient towards a target site; positioning the medical device at the target site; placing a fixation device at a subcutaneous location within the patient, the fixation device including a catheter receiving portion defining a cavity and a pair of wings; and clamping the pair of wings to secure the catheter within the catheter fixation portion. In some aspects, the medical device may be an intracardiac pump and the target site may be across a native heart valve.

The method may further include creating an incision through skin of the patient adjacent a clavicle. The method may also include securing the fixation device to a native or artificial portion of the cardiovascular system of the patient. For example, the native or artificial portion of the cardiovascular system of the patient may be a graft sutured to an axillary artery of the patient.

In additional aspects, the placing step may further include placing the catheter receiving portion about a portion of the graft. Thus, the clamping step and the securing steps may be performed simultaneously, for example, by stapling the pair of wings together. In one aspect, an inner layer of the fixation device may include a resilient material, and the clamping step, may include compressing the inner layer around the catheter.

### BRIEF DESCRIPTION OF THE DRAWINGS

Various embodiments of the present disclosure are described herein with reference to the drawings, wherein:

FIG. 1 is a highly schematic cutaway view of the human heart, showing a percutaneous axillary artery puncture approach for delivering an intracardiac heart pump across the aortic valve;

FIG. 2 is perspective view of an intracardiac heart pump and sheath assembly according to an embodiment of the present disclosure;

FIG. 3 is a highly schematic cutaway view of the human showing the intracardiac heart pump of FIG. 2 properly positioned across the native aortic valve;

FIG. 4 is an elevation view of a subcutaneous catheter fixation in an unclamped condition and in accordance with an embodiment of the present disclosure;

FIG. 5 is a plan view of the subcutaneous catheter fixation of FIG. 4; and

FIG. 6 is an elevation view of the subcutaneous catheter fixation of FIG. 4 in a clamped condition.

### DETAILED DESCRIPTION

Intracardiac pump assemblies can be introduced into the heart either surgically or percutaneously and used to pump blood from one location in the heart or circulatory system to another location in the heart or circulatory system. When deployed in the heart, for example, an intracardiac pump can transfer blood from the left ventricle to the aorta, or from the inferior vena cava to the pulmonary artery. Traditionally, intracardiac pumps operate in parallel with the native heart to supplement cardiac output and partially or fully unload components of the heart. For this reason, intracardiac pumps are often utilized in instances of cariogenic shock, during high-risk percutaneous coronary intervention (PCI), right heart failure, congestive heart failure, or severe lung failure, to relieve stress on the heart during recovery or while the patient awaits a heart transplant. Examples of such systems include the IMPELLA® family of devices designed by Abiomed, Inc., Danvers Mass.

Several IMPELLA® devices are of relatively small circumferential size and can be percutaneously delivered into a patient less invasively than intracardiac pumps that are implanted during traditional, full open-chest surgery. When delivered percutaneously, these intracardiac pumps may be inserted into a patient and delivered via a tube-like delivery device such as a catheter.

During delivery, the catheter is tracked through the vasculature of a patient to advance the intracardiac pump to a target site (e.g., across the aortic valve in the case of “left-side” intracardiac devices, or across the pulmonary valve in the case of “right-side” intracardiac devices). Echocardiography, fluoroscopy, and/or other imaging techniques may be utilized during delivery to properly position the intracardiac pump. Once in position, the intracardiac pump may be turned on to suction blood through an inflow portion (e.g., a blood inflow cage) of the intracardiac device, and to expel the pumped blood from an outflow portion (e.g., a blood outflow cage) of the intracardiac device. In the case of left-side intracardiac pumps, the blood inflow cage of the intracardiac device may be positioned within the left ventricle of the heart, and the blood outflow cage of the intracardiac pump may be positioned within the aorta. On the other hand, in the case of right-side intracardiac pumps, the blood inflow cage of the intracardiac device may be positioned within the inferior vena cava, and the blood outflow cage of the intracardiac device may be positioned within the pulmonary artery.

The clinical success of intracardiac pumps is dependent, in part, on proper positioning of the pump. For example, in the case of left-side intracardiac pumps, if the blood inflow cage is not properly positioned within the left ventricle, and without obstruction from anatomical structures such as the ventricular wall or the mitral valve, the intracardiac pump will inefficiently suction blood from the left ventricle. Additionally, if the blood outflow cage of the intracardiac pump is not sufficiently positioned within the aorta, the pumped blood may return to the left ventricle, causing further stress on the heart. Thus, it is important for intracardiac pumps to be stabilized in the proper position throughout the entire treatment. For this reason, delivery devices used in percutaneous intracardiac pump procedures, often include a butterfly structure that can be secured to the skin of a patient and a securement device, such as a conventional Tuohy-Borst type device, to prevent the catheter from moving after the intracardiac pump has been properly positioned.

Despite the improvements that have been made to intracardiac pumps and associated delivery assemblies, shortcomings remain. For example, the inventors have recognized that even when the assembly is sutured or otherwise secured to the skin of the patient, ambulation or other movement of the patient, can alter the position of the catheter relative to the anatomy of the patient and/or the access site, and cause the intracardiac pump to migrate from the desired position (i.e., become mispositioned). In some instances, such movement may even damage the sutures, resulting in further movement of the catheter. Accordingly, the inventors have recognized and appreciated the numerous benefits associated with the catheter fixation devices disclosed herein that are configured to clamp the catheter to a graft and prevent the catheter from migrating relative to the patient anatomy and/or access site.

When deployed in the heart, an intracardiac device collects blood from one area of the heart and pumps the blood to another area of the heart, to assist the heart in performing its normal function. As used herein in connection with an intracardiac pump, the term “inflow” refers to the portion of the intracardiac pump through which blood enters the pump, and the term “outflow” refers to the portion of the intracardiac pump through which blood is expelled. When used in connection with devices for delivering the intracardiac pump into a patient, the terms “proximal” and “distal” are to be taken as relative to the user of the delivery devices. For example, “proximal” or “proximal end” is to be understood as relatively close to the operator, and “distal” or “distal end” is to be understood as relatively farther away from the operator. Also as used herein, the terms “substantially,” “generally,” “approximately,” and “about” are intended to mean that slight deviations from absolute are included within the scope of the term so modified.

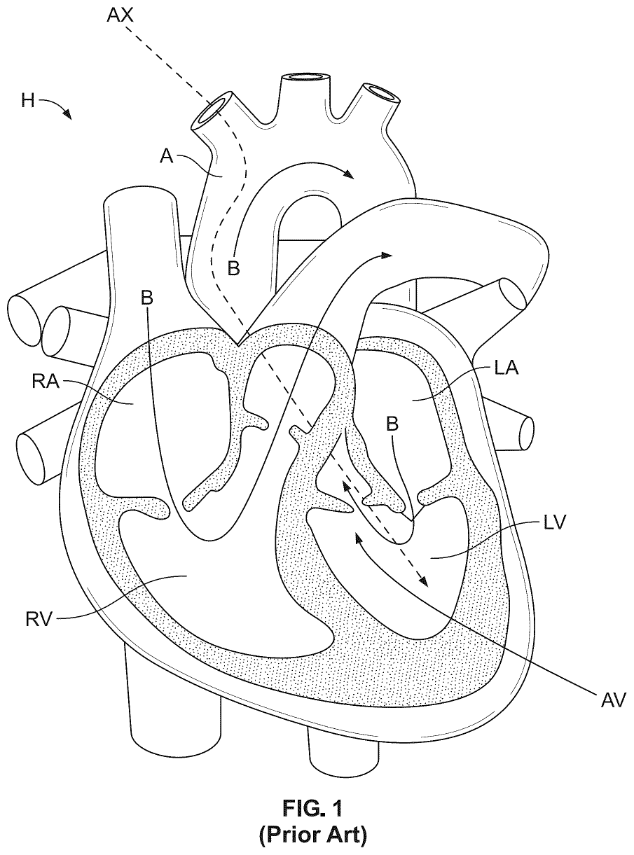

FIG. 1 is a schematic cutaway representation of a human heart H. The human heart includes two atria and two ventricles: right atrium RA and left atrium LA, and right ventricle RV and left ventricle LV. Heart H further includes aorta A. Disposed between left ventricle LV and aorta A is aortic valve AV. The aortic valve, also known as the left semilunar valve or the left arterial valve, generally includes three leaflets that coapt to regulate blood flow between left ventricle LV and aorta A. When left ventricle LV contracts during systole, aortic valve AV opens, and blood is pushed from the left ventricle through aorta A to major arteries of the vasculature system. Blood flows through heart H in the direction shown by arrows “B”.

A dashed arrow, labeled “AX”, indicates an axillary artery puncture, an approach of delivering an intracardiac pump to a target site, in this case to a location across aortic valve AV. Using an axillary artery puncture, the intracardiac pump is inserted into the axillary artery through an infraclavicular incision and then introduced to the target site via aorta A. Echocardiography, fluoroscopy, and/or other imaging techniques may be used to help guide a delivery device, such as a catheter, to the target site. Other approaches are possible for delivering the intracardiac pump across aortic valve AV, such as a direct aortic approach, or to other target sites within heart H.

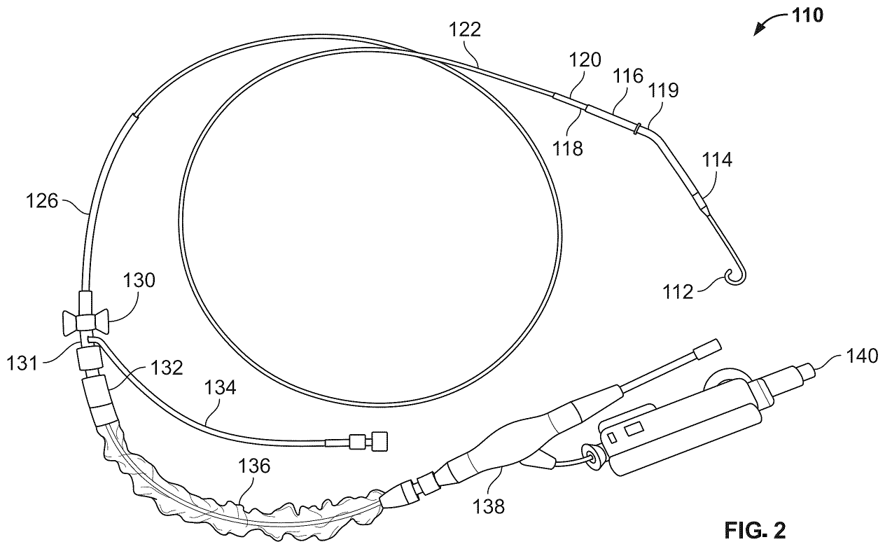

FIG. 2 illustrates a intracardiac pump and sheath assembly 110. A handle 138 is provided at the proximal end of intracardiac pump and sheath assembly 110. Handle 138 may be operably coupled to the proximal end of a catheter 122 and, therefore, arranged to advance the catheter within the cardiovascular system of a patient and to retract the catheter from the cardiovascular system of the patient, unless movement of the catheter is prevented by a securement device 132, as will be explained in further detail hereinafter.

Catheter 122 may be enclosed by a protective sleeve 136 extending between handle 138 and securement device 132. Put differently, the proximal end of protective sleeve 136 may be attached to handle 138, and the distal end of the protective sleeve may be attached to securement device 132, to enclose and protect a proximal portion of catheter 122. Protective sleeve 136 may be formed of any material, such as a medical grade plastic, suitable for preventing the proximal end of catheter 122 from being contaminated as the catheter is advanced into the vasculature of a patient.

A hemostasis valve 131 may be provided between a distal end of securement device 132, and a proximal end of butterfly 130. Hemostasis valve 131, securement device 132, and butterfly 130 may be removably coupled from one another or manufactured as a single unitary component. A proximal end of a sheath 126 is coupled to butterfly 130. Sheath 126 defines a lumen extending from the proximal end of the sheath to a distal end of the sheath that is sized to slidably receive catheter 122 therethrough.

Intracardiac pump and sheath assembly 110 may also include a purge fluid port 140. The purge fluid port 140 is in communication with handle 138 and is arranged to provide purge fluid (e.g., a solution of dextrose or glucose with heparin) to a purge lumen (not shown) within catheter 122. The purge lumen of catheter 122 is in fluid communication with, and configured to deliver the purge fluid to, the motor housing 120 of the intracardiac pump. Motor housing 120 may include a motor and an impeller. In some embodiments, the motor may be external to the patient, in which case catheter 122 may enclose a flexible drive, such as a shaft or cable, and motor housing 120 may enclose an impeller connected to that drive shaft or cable.

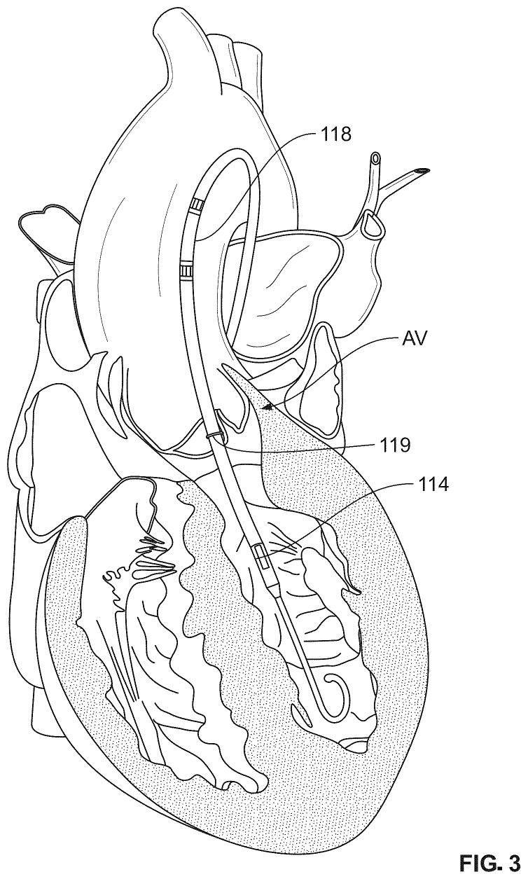

With continued reference to FIG. 2, the distal end of motor housing 120 may be coupled to a blood outflow cage 118. The distal end of blood outflow cage 118 may be coupled to a cannula 116, which in turn is coupled a proximal end of blood inflow cage 114. Cannula 116 may include a marking 119, such as a radiopaque marker that is visible under fluoroscopy, to assist a clinician in properly positioning the cannula within the native heart valve as shown in FIG. 3. In some embodiments, the cannula 116 may be expandable. In some instances, an atraumatic extension, such as pigtail extension 112, may optionally extend from the distal end of blood inflow cage 114. In this regard, when left ventricle LV contracts during systole, the left ventricular wall may contact pigtail extension 112, instead of blood inflow cage 114, thereby preventing intracardiac pump from damaging the ventricular wall. In other instances, the blood pump may not include a pigtail extension 112.

When the pump is operated, blood will be pumped in the proximal direction from blood inflow cage 114, through connecting cannula 116, to blood outflow cage 118. In this respect, the intracardiac pump illustrated in FIG. 2 is designed for left heart support. The intracardiac pump, however, may alternatively be configured to pump blood in the distal direction (e.g., for applications where the pump is used for right heart support), in which case cage 118 would operate as the blood inflow cage, and cage 114 would operate as the blood outflow cage.

Intracardiac pump and sheath assembly 110 may be introduced into the vasculature of a patient via an introducer sheath assembly (not shown). In other instances, the intracardiac pump may be directly inserted into vasculature of the patient, or through a graft sutured to the cardiovascular system of a patient, in a chimney fashion. When delivering an intracardiac pump using a percutaneous axillary artery approach, intracardiac pump and sheath assembly 110 is more commonly inserted through a graft (e.g., without an introducer sheath assembly).

Once sheath 126 has been fully inserted into the patient, the clinician may secure the sheath to the patient at or near the incision (e.g., adjacent the clavicle) using butterfly 130. In this regard, butterfly 130 may be affixed to the patient, using adhesives or sutures, to secure the sheath 126 and securement device 132 relative to the patient. With sheath 126 secured to the patient, the clinician may then advance the intracardiac pump to the target site and use securement device 132 to restrict further movement of the intracardiac pump after it has been properly positioned within heart H.

Securement device 132 may be a conventional Tuohy-Borst type device, although any other securement device known in the art may be used. Conventional Tuohy-Borst type devices include a barrel that may be rotated in a first direction (e.g., a clockwise direction) to clamp securement device 132 about catheter 122, which in turn restricts movement of the catheter. As mentioned herein, however, movement of the patient can adjust the positioning of catheter 122 between securement device 132 and the distal end of the catheter, which in turn, can cause migration of the intracardiac pump.

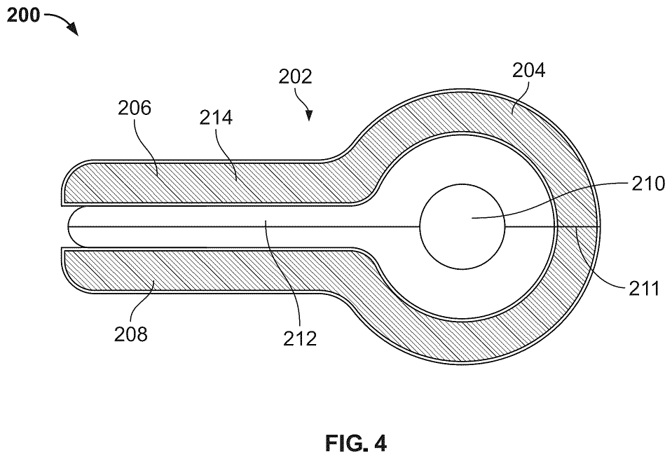

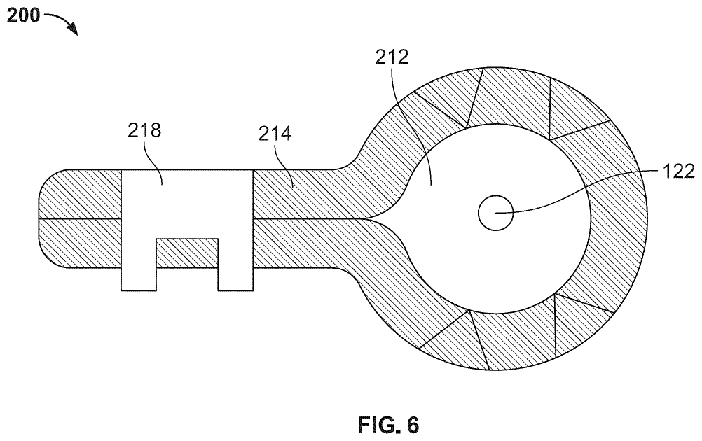

FIGS. 4-6 illustrate a catheter fixation device 200 (sometimes referred to herein simply as “a fixation device”) that is arranged to be implanted subcutaneously to clamp catheter 122 and secure the catheter within the cardiovascular system of the patient. It will be appreciated that the intracardiac pump will be more stably secured within heart H as the distance between catheter fixation device 200 and the heart decreases. That is, reducing the length between the intracardiac pump and the location at which the catheter is fixed, will reduce the ability of the intracardiac pump to migrate. For this reason, securing catheter 122 to a graft, or subcutaneous anatomy proximate the heart, such as the axillary artery, will improve stability of the intracardiac pump compared to conventional external securement devices.

Herein, catheter fixation device 200 is primarily described in conjunction with intracardiac pump and sheath assembly 110. More specifically, in some embodiments, fixation device 200 may replace the structure and functionality of butterfly 130 and securement device 132. In some embodiments, catheter fixation device 200 may be used with intracardiac pump and sheath assembly 110 as depicted in FIG. 2 (i.e., along with butterfly 130 and securement device 132). It will be appreciated that catheter fixation device 200 may be used with any intracardiac blood pump, or other medical device, delivered into a patient by a delivery system, such as a catheter, when a clinician wishes to selectively restrict movement of that device. Examples of such systems include angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, or any other venous or arterial based introduced catheters and devices.

Catheter fixation device 200 may include a body 202 formed of a biocompatible material, a catheter receiving portion 204, and first and second wings 206, 208 that are designed to be clamped. Catheter receiving portion 204 may define a cavity 210 for receiving a catheter such as catheter 122. Cavity 210 may have a generally circular cross-section that sized to substantially match, or that is slightly larger than, a cross-section of catheter 122 when first and second wings 206, 208 are in the unclamped condition. In this regard, cavity 210 may be sized to slide around a graft sewn to the native cardiovascular system of the patient and designed to reduce in size when first and second wings 206, 208 are clamped to secure catheter 122 within the catheter receiving portion. In other aspects, cavity 210 may have alternative cross-sectional shapes, so long as catheter receiving portion 204 is designed to selectively compress and fixate catheter 122 when first and second wings 206, 208 are clamped.

The body 202 of fixation device 200 may optionally define a hinge 211 provided at a mid-line of the body. In this regard, first and second wings 206, 208 may pivoted away from one another, thereby allowing catheter 122 to be inserted between the wings and into cavity 210. Body 202 may include an inner layer 212 and an outer layer 214 formed of different materials for performing different function. For example, inner layer 212 may be formed of a substantially resistant material, such as a polymer, and more particularly a rubber or a silicone.

In this regard, the inner layer 212 of receiving portion 204 may designed to compresses catheter 122, thereby preventing movement of the catheter, while distributing the compressible load to prevent damage. However, any suitable material may be used for the inner layer 212.

In some examples, the innermost surface of the inner layer 212 may be textured to increase friction and improve catheter fixation. In some embodiments, the thickness of the inner layer 212 of the first and second wings 206, 208 may be different than the thickness of the inner layer 212 of the catheter receiving portion 204. For example, the thickness of the catheter receiving portion 204 may be greater than the thickness of the inner layer of the first and second wings 206, 208. As will be appreciated, any suitable number of layers may be used. In such examples, one or more layers may be made of the same material.

Outer layer 214, on the other hand, may be formed of a substantially rigid material, such as stainless steel, or another medical grade metal or metal alloy. The substantially rigid material of outer layer 214 may provide and maintain a clamping force on the catheter after first and second wings 206, 208 have been transitioned to the clamped condition. Nevertheless, the materials from which the body 202 of fixation device 200 is formed is not limited thereto. Fixation device 200 may alternatively be formed of different materials, a single material, or even more than two materials.

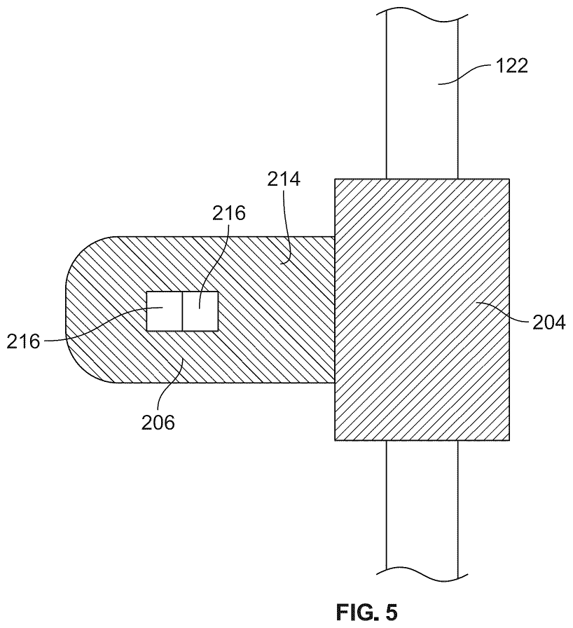

First and second wings 206, 208 may extend from catheter receiving portion 204 in the same direction and in parallel planes. With specific reference to FIG. 5, the substantially rigid outer layer 214, may define one or more apertures 216 for receiving a securement device such as a surgical staple 218, a suture, a biocompatible adhesive, or any other suitable means. When stapled, the staple is designed to pass through the aperture(s) 216 to clamp first and second wings 206, 208, which in turn, may compress catheter receiving portion 204 and may secure the substantially resilient inner material about catheter 122.

In use, catheter fixation device 200 may be used in conjunction with an intracardiac pump and sheath assembly 110 to efficiently stabilize the intracardiac pump within heart H. Although use of catheter fixation device 200 is described hereinafter in connection with a percutaneous intracardiac pump that is delivered to the left-side of the heart using an axillary approach, it will be appreciated that the catheter fixation device 200 may be used in conjunction with percutaneous intracardiac devices providing right heart support, as well as other medical devices secured to a delivery system (e.g., a catheter) for which a clinician wishes to selectively restrict movement.

First, a physician may make an infraclavicular incision to provide an access point to the axillary artery of the patient. One end of a graft, such as a chimney graft, may then be sutured to the axillary artery. With the graft secured to the axillary artery, the physician may pivot the first and second wings 206, 208 away from one another and about hinge 211, and insert the graft between the wings and into the cavity 210 of receiving portion 204. Alternatively, in embodiments in which catheter fixation device 200 does not include hinge 211, an end of the graft may be inserted through the cavity 210 of catheter receiving portion 204 as the fixation device is slid along an external surface of the graft towards the axillary artery. The physician may optionally repeat this process to place additional catheter fixation devices 200 along the length of the graft for additional stability if desired. Next, the intracardiac pump may then be secured to the distal end of intracardiac pump and sheath assembly 110, advanced through the graft, and into the axillary artery.

Under echocardiography and/or other traditional imaging techniques, the clinician may operate handle 138 to advance catheter 122 toward the target site by tracking the intracardiac pump into heart H via aorta A. As shown in FIG. 3, while using fluoroscopy, the physician may align the marking 119 on connecting cannula 116 within the aortic valve AV for deployment in the left ventricle of a patient. In this position, blood inflow cage 114 may be centrally positioned within left ventricle LV and blood outflow cage 118 may be sufficiently positioned within aorta A.

After the clinician has confirmed that the intracardiac pump is properly positioned within the heart H of the patient, a surgical staple 218 may be inserted through aperture(s) 216 to clamp first and second wings 206, 208. As first and second wings 206, 208 are clamped together, the rigid of outer layer 214 may compress the inner layer 212, thereby reducing the circumferential size of catheter receiving portion 204 to compresses the graft around catheter 122. In this regard, when first and second wings 206, 208 are clamped, catheter fixation device 200 may be simultaneously secured to catheter 122 and the graft. At the same time, catheter 122 may be stabilized within the catheter receiving portion 204 and relative to the cardiovascular system of the patient. Alternatively, a clinician may use a suture to clamp first and second wings 206, 208. However, it will be appreciated that stapling may expedite the surgical procedure and remove user variability, compared to suturing.

In other aspects, when a graft is not used to introduce intracardiac pump and sheath assembly 110 into the cardiovascular system of the patient, the intracardiac blood pump may be introduced directly into patient anatomy. In such instances, the catheter fixation device 200 may be used to directly clamp and stabilize catheter 122 within the catheter receiving portion 204 of the fixation device. In these instances, the catheter fixation device 200 must also be stapled, or otherwise secured, to a native or artificial subcutaneous structure to immobilize the fixation device itself, thereby stabilizing the catheter within the cardiovascular system of the patient. For example, the catheter fixation device 200 may be secured to a patient's fascia. It will be appreciated that such fixation may be performed simultaneously with the clamping of the first and second wings 206, 208, or as an additional step, either before or after the wings are clamped.

In other aspects when a graft is not used to introduce intracardiac pump, the intracardiac pump may be introduced temporally via the sheath assembly 110 as discussed herein and the catheter fixation device 200 may be used to directly clamp onto the catheter 122 on the proximal side of the sheath assembly 110. In such instances, the catheter is stabilized and secured relative to the sheath assembly 110 because the catheter is secure relative to the catheter receiving portion 204 of the catheter fixation device 200.

With the intracardiac pump stabilized across the aortic valve AV, the intracardiac pump may be turned on to suction blood from the left ventricle LV into blood inflow cage 114, through connecting cannula 116, and out from blood outflow cage 118 into aorta A. Thereafter, the incision may then be closed.

As disclosed herein, catheter fixation device 200 offers several advantageous over traditional securement devices such as securement device 132. A previously mentioned, when catheter 122 is externally fixed, movement of the patient may result in movement of the catheter, which in turn may result in migration of the intracardiac pump. Stabilizing the catheter fixation device 200 subcutaneously (e.g., closer to the heart) reduces catheter 122 motion due to patient movement.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A catheter fixation device, comprising:
a body arranged to be subcutaneously secured within a patient, the body including a catheter receiving portion defining a cavity, and first and second wings extending from the catheter receiving portion,
wherein the first and second wings are moveable relative to one another from an unclamped position, in which a catheter is moveable within the catheter receiving portion, and a clamped position, in which the catheter is fixed within the catheter receiving portion.

2. The device of claim 1, wherein the body is formed of a biocompatible material and the body includes an inner layer comprising a substantially resilient material and an outer layer comprising a substantially rigid material.

3. The device of claim 1, wherein the substantially resilient material is a polymeric material.

4. The device of claim 2, wherein the outer layer comprises a metal or metal alloy.

5. The device of claim 2, wherein the outer layer defines one or more apertures arranged to receive a closure device.

6. The device of claim 5, wherein the closure device is a staple.

7. A sheath assembly, comprising:
a catheter for delivering a medical device into a cardiovascular system of a patient, and
a catheter fixation device including a body formed of a biocompatible material arranged to be subcutaneous secured within the patient, the body including a catheter receiving portion defining a cavity, and first and second wings extending from the catheter receiving portion,
wherein the first and second wings are moveable relative to one another from an unclamped position, in which the catheter is moveable within the catheter receiving portion, and a clamped position, in which the catheter is fixed within the catheter receiving portion.

8. The assembly of claim 7, further comprising the medical device, the medical device being an intracardiac pump.

9. The device of claim 7, wherein the body includes an inner layer comprising a substantially resilient material and an outer layer comprising a substantially rigid material.

10. The device of claim 9, wherein the substantially resilient material comprises a polymeric material, and wherein the substantially rigid material comprises a metal or metal alloy.

11. The device of claim 10, wherein the outer layer defines an aperture arranged to receive a staple.

12. A method of stabilizing a medical device within a cardiovascular system of a patient, the method comprising step of:
tracking a catheter, carrying a medical device, through the cardiovascular system of the patient towards a target site;
positioning the medical device at the target site;
placing a fixation device at a subcutaneous location within the patient, the fixation device including a catheter receiving portion defining a cavity and a pair of wings; and
clamping the pair of wings to secure the catheter within the catheter fixation portion.

13. The method of claim 12, wherein the medical device is an intracardiac pump and the target site is across a native heart valve.

14. The method of claim 12, further comprising:
creating an incision through skin of the patient adjacent a clavicle of the patient.

15. The method of claim 12, further comprising securing the fixation device to a native or artificial subcutaneous structure.

16. The method of claim 15, wherein the native or artificial subcutaneous structure is a graft sutured to an axillary artery of the patient.

17. The method of claim 16, wherein the placing step further comprises placing the catheter receiving portion about a portion of the graft.

18. The method of claim 15, wherein the clamping step and the securing steps are performed simultaneously.

19. The method of claim 18, wherein the clamping step and the securing step are simultaneously performed by stapling the pair of wings together.

20. The method of claim 12, wherein an inner layer of the fixation device includes a resilient material, and the clamping step, comprises:
compressing the inner layer around the catheter.
